(19) United States
(12) Patent Application Publication
(10) Pub. No.: US 2022/0296450 A1
(43) Pub. Date: Sep. 22, 2022

(54) LIMB SUPPORT FOR MEDICAL PROCEDURES

(71) Applicant: Main Line Health, Inc., Bryn Mawr, PA (US)

(72) Inventor: Colleen Rogers, Jenkins Twp, PA (US)

(73) Assignee: Main Line Health, Inc., Bryn Mawr, PA (US)

(21) Appl. No.: 17/832,198

(22) Filed: Jun. 3, 2022

Related U.S. Application Data
(63) Continuation of application No. PCT/US21/12505, filed on Jan. 7, 2021.
(60) Provisional application No. 62/958,125, filed on Jan. 7, 2020.

(51) Int. Cl. A61G 13/12

(52) U.S. Cl. CPC A61G 13/129; A61G 13/126; A61G 13/1295

(57) ABSTRACT

A height-adjustable support for supporting various bodily limbs over a wide range of limb positions, and in hospital rooms, doctors' offices, or other compact environments. The device permits a medical procedure to be performed by a single person while the device supports the limb. The device includes a rigid base, at least one support mounted to the base to extend therefrom, and a cradle mounted on the support, the cradle defining a surface configured to support a limb of a patient. The supports may be adjustable in length, and the cradle may be adjustable in angular position. The cradle may be U-shaped or otherwise concavely-shaped, and may be linked with a cushioning material. A cover may be provided, and the cover and/or cradle may be configured with complementary fasteners for releasably attaching the cover to the cradle, so that the cover may be discarded after each use.

### CROSS-SECTION TO RELATED APPLICATION

This application claims the benefit of priority, under 35 U.S.C. § 119(e), of U.S. provisional patent application No. 62/958,125, filed Jan. 7, 2020, the entire disclosure of which is hereby incorporated herein by reference.

### FIELD OF THE INVENTION

The present invention relates generally to limb supports for medical and surgical procedures, and more particularly, relates to a simple, mobile, height-adjustable support suitable for use for various different limbs over a range of limb positions, and in hospital rooms or in other compact environments in which space is limited.

### DISCUSSION OF RELATED ART

Various supports are known in the prior art for holding in place the limb of a person. Some of these devices are not height and/or position adjustable, limiting their usability. Others are large and bulky, and unsuitable for use in compact environments in which space is limited. Some of these devices are permanently attached to an operating table or bed, and thus are not mobile, and are positioned for dedicated use to support an arm, or a leg, but not both. Some of these devices have structure limiting how the device can support the limb. Some of these devices are mechanically complex, and expensive.

What is needed is a simple, mobile, height-adjustable support suitable for use for various different limbs over a range of limb positions, and in hospital rooms or other compact environments in which space is limited.

### SUMMARY

The present invention provides a simple, mobile, height-adjustable support suitable for use for various different limbs over a range of limb positions, and in hospital rooms, doctors' offices, or other compact environments in which space is limited. Such a device requires only a single person to operate it, and it permits an associated medical procedure to be performed by a single person, thereby eliminating the need for a second person for holding and supporting the limb during a medical procedure, as is commonplace. Further, the device provides several advantages relative to various prior art devices, including that the device is mobile and easily transported/moved to a position alongside the patient, and that the device can be used while the patient is in a wheelchair, chair or bed. Further, use of the device ensures that there is less strain on the patient, and helps the patient to feel more secure and comfortable with the patient's limb in the device, which affords the caregiver ample time to evaluate the limb/wound and/or to perform the medical procedure properly while providing comfort to the patient.

### BRIEF DESCRIPTION OF THE FIGURES

An understanding of the following description will be facilitated by reference to the attached drawings, in which:

FIG. 1 is a perspective view of a limb support device in accordance with an exemplary embodiment of the present invention;

FIG. 2 is a perspective view of the limb support of FIG. 1, shown in use to support an arm of a patient in a hospital bed;

FIG. 3 is a perspective view of the limb support of FIG. 1, shown in use to support a leg of a patient in a hospital bed;

FIG. 4 is a perspective view of a disposable cover for use with the limb support device of FIG. 1;

FIG. 5 is a perspective view of the limb support of FIG. 1, shown positioned to make a foot accessible for treatment of a foot wound; and

FIG. 6 is a perspective view of the limb support of FIG. 1, shown positioned to make a leg accessible for treatment of a leg wound.

### DETAILED DESCRIPTION

The present invention provides a simple, mobile, height-adjustable support suitable for use for various different limbs over a range of limb positions, and in hospital rooms, doctors' offices, or other compact environments in which space is limited. Such a device requires only a single person to operate it, and it permits an associated medical procedure to be performed by a single person, thereby eliminating the need for a second person for holding and supporting the limb during a medical procedure, as is commonplace. Further, the device provides several advantages relative to various prior art devices, including that the device is mobile and easily transported/moved to a position alongside the patient, and that the device can be used while the patient is in a wheelchair, chair or bed. Further, use of the device ensures that there is less strain on the patient, and helps the patient to feel more secure and comfortable with the patient's limb in the device, which affords the caregiver ample time to evaluate the limb/wound and/or to perform the medical procedure properly while providing comfort to the patient.

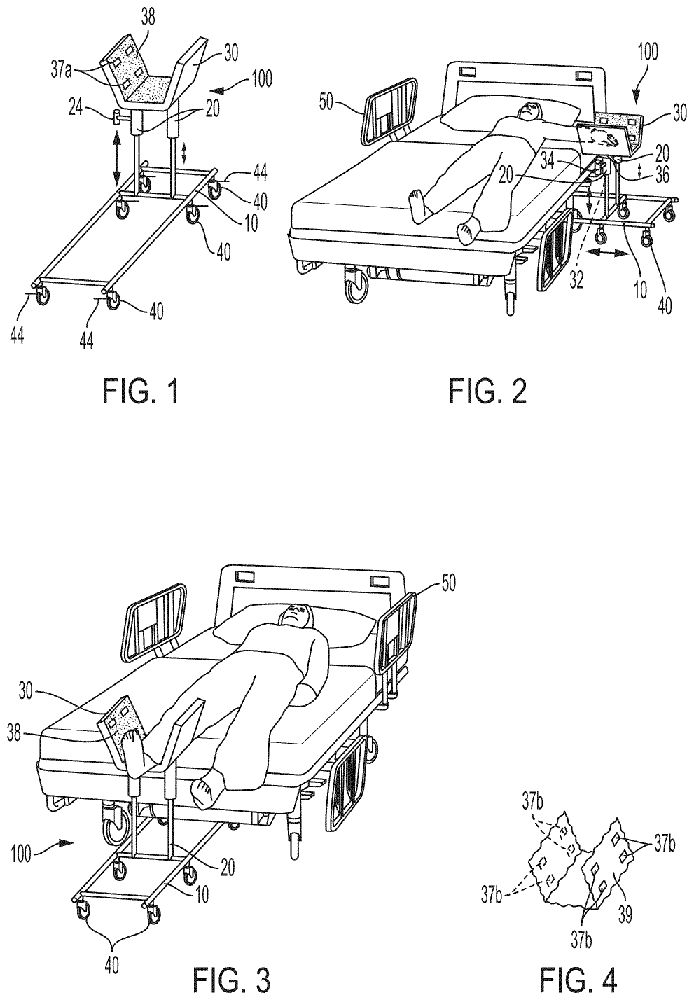

Referring now to FIG. 1, a limb support device 100 in accordance with an exemplary embodiment of the present invention. As will be noted from FIG. 1, the exemplary limb support device 100 is a free-standing device separate and apart from any hospital bed.

As will be appreciated from FIG. 1, the limb support device 100 includes a base 10, one or more adjustable supports 20, and a cradle 30. The base 10 includes a rigid frame supporting at least one adjustable support 20. In certain embodiments, the device 100 includes two or more adjustable supports 20. The adjustable support(s) 20 are preferably eccentrically located, e.g. on less than one half of the length of the base as shown in FIG. 1, so that the cradle is not supported centrally along the length of the base 10. Accordingly, a portion of the base 10, namely, the portion in which the adjustable support(s) is/are not located, is adapted to extend well beneath a bed (or chair). This arrangement provides stability to the support device 100 while also facilitating use of the support device in hospital rooms or other compact environments, with at least a portion of the base 10 positioned beneath a bed/chair while the cradle is positioned adjacent the bed/chair for use to support a limb of the patient.

The adjustable supports 20 may have any suitable form permitting vertical adjustment of the length of the supports 20, so that the cradle may be selectively positioned upwardly and downwardly, and then be secured in place to fix its position vertically. By way of example, the adjustable supports 20 may include pressurized hydraulic or pneumatic cylinders that may be used to drive movement of the cradle 30 relative to the base 10, and that are selectively lockable/unlockable by way of a release lever 24, as known in the art. Various suitable mechanisms for providing such height adjustability are known in the art, and any suitable mechanism may be used. Preferably, the adjustable support(s) 20 provide(s) for adjustment of a base portion of the cradle 30 from about 12″ above the floor (e.g., for supporting the foot of a patient seated in a wheelchair) to about 48″ above the floor (e.g., a comfortable height for a standing nurse and a patient in an elevated bed), or within any sub-range thereof, to allow for accommodation of a limb at a range of heights likely to be encountered as the patients sits in a chair or lies in a hospital bed.

The cradle 30 is preferably generally concave in shape (open upwardly), and may be U-shaped, and oriented with the open end of the “U” facing upwardly, to facilitate receipt and support of a patient's limb, as best shown in FIGS. 1 and 3. The cradle 30 is preferably elongated longitudinally, in the same direction of elongation as the base 10, as best shown in FIGS. 1 and 2, and may have a uniform or substantially uniform cross-section along its length, as shown in FIG. 1. In one embodiment, the cradle 30 is movably mounted relative to the base 10 and/or the supports 20, to allow the angle of the cradle 30 to be adjusted to corresponding to a comfortable, or otherwise desirable, angle of the associated limb to be placed in the cradle 30. Any suitable mechanism may be used for this purpose. By way of example, the cradle 30 may be pivotably supported on the support(s) 20 by way of a bolt 32 and a nut 34 incorporated into a lever 36 that is manually operable to loosen and tighten the nut to selectively lock the cradle 30 into a desired angular position, as best shown in FIG. 2.

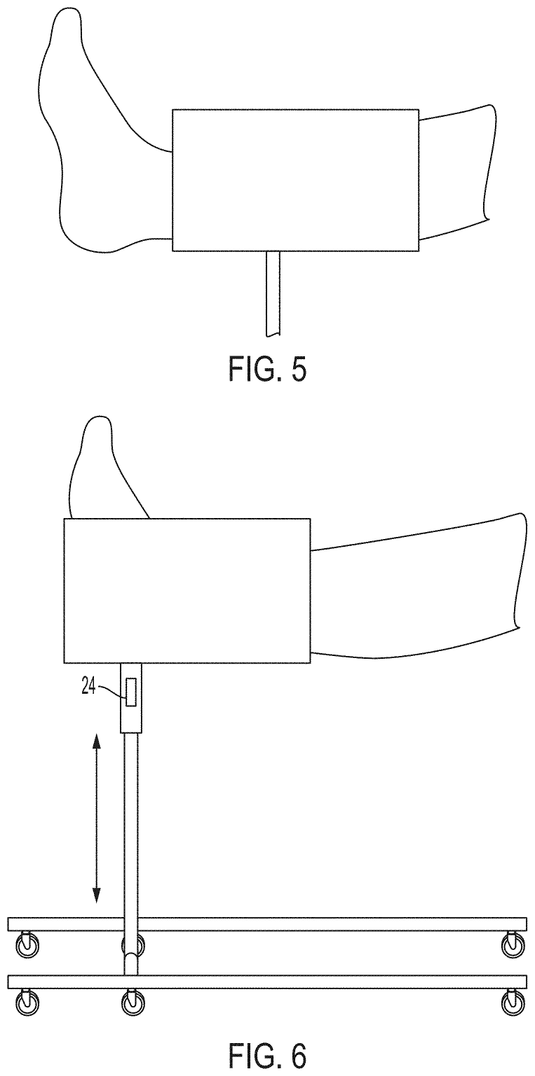

Optionally, an interior surface of the cradle 30 may be lined with a cushioning liner 38. The liner may be made of a cushioning material, such as lambswool, foam rubber or the like, to increase the comfort of the patient. Additionally, the cradle/liner may be intended to be used in conjunction with a disposable cover 39, as shown in FIG. 4. The disposable cover 39 is intended for a single use/single patient only, such that the cover is disposable, and replaced before each new patient is treated to prevent cross-contamination of patients. In such an embodiment, the cradle/liner and/or the cover may be provided with complementary releasable fasteners 37a, 37b, such as fields of hook-and-loop fastener, to allow the cover to be releasably mounted to the cradle, to cover the cushioning material, and then to be easily removed and disposed of after use/before a next user, as best shown in FIGS. 1 and 4. By way of example, the disposable cover may be made be provided in the form of a thin sheet of paper, plastic film, etc.

If desired, wheels or casters 40 may be secured to the base to assist in moving the device 100 to various positions adjacent a bed, etc., as may be desired. One or more, and preferable at least two, of the wheels/casters 40 may be selectively lockable to prevent theft rotation via a lock lever 44. Such wheels/casters 40 are well-known in the art.

Accordingly, a single instance of the device 100 is movable/mobile, and positionable as desired relative to a hospital bed (or chair), and separately from a hospital bed (or chair) for use to support various different limbs of a person/patient, such as a left arm, right arm, left leg or right leg, as desired. FIG. 2 shows the limb support device 100 of FIG. 1 beside a hospital bed 50, in position for use to support an arm/hand of a patient laying in the bed 50. FIG. 3 shows the same limb support device 100 at the foot of a hospital bed 50, in position for use to support a leg/foot of a patient laying in the bed 50. In both cases, a portion of the base 10 is positioned beneath the bed 50, which is advantageous in compact environments in which available floorspace is limited, as is commonly the case in a typical patient-stay hospital room. Repositioning of the limb support device 100 is facilitated by its wheels/casters 40. FIG. 5 shows the limb support device positioned with the cradle 30 relatively near to the bed, to position the cradle 30 such that it engages and supports the leg, and makes the foot accessible for treatment of a foot wound. FIG. 6 shows the limb support device positioned with the cradle 30 relatively far from the bed, to position the cradle 30 such that it engages and supports the foot, and makes the leg accessible for treatment of a foot wound.

In use, the limb support device 100 may be positioned adjacent a bed or chair in which the patient is resting. This may involve unlocking the wheels/casters 40 using the lock lever 44 and rolling the device 100 into position adjacent the bed/chair, and operating the lock lever 44 to lock the wheels in place. The adjustable supports 20 may then be operated to raise or lower the cradle 30 to a desired height for comfortably receiving and supporting a limb of the patient as the patient remains in the bed/chair. This may involve manual operation of the release lever 24. The angular orientation of the cradle may be similarly adjusted. This may involve manual operation of the lever 36. A disposable cover 39 may be applied on the cradle/over the cushioning liner 38, by mating fasteners 37b, 37a of the cover 39 and liner 38, to provide a sanitized/clean/fresh surface. After the wheels and/or adjustable supports 20 are suitably adjusted, and a cover 39 provided, if desired, the patient's limb may be placed in the cradle 30, and may be reliably supported by the limb support device 100 in a handsfree fashion, freeing the hands of a healthcare provider to perform a medical/surgical procedure, and without requiring assistance of another healthcare provider to support the limb during the medical/surgical procedure. After the procedure has been completed, the fasteners 37a, 37b may be decoupled, and the cover 39 may be discarded, to ready the limb support device 100 for subsequent use with another patient.

While there have been described herein the principles of the invention, it is to be understood by those skilled in the art that this description is made only by way of example and not as a limitation to the scope of the invention.

Accordingly, it is intended by the appended claims, to cover all modifications of the invention which fall within the true spirit and scope of the invention.

What is claimed is:

1. A limb support device comprising:
a rigid base;
at least one support mounted to said base to extend therefrom; and
a cradle mounted on said at least one support, said cradle defining a surface configured to support a limb of a patient.

2. The limb support device of claim 1, wherein said at least one support is adjustable in length.

3. The limb support device of claim 2, wherein said at least one support comprises one of a hydraulic cylinder and a pneumatic cylinder.

4. The limb support device of claim 2, wherein said at least one support comprises a release lever operable to selectively permit adjustment of the length of said at least one support, and to selectively fix the length of said at least one support.

5. The limb support device of claim 4, further comprising at least one wheel supported on said base and extending in a direction opposite to that of said at least one support.

6. The limb support device of claim 5, wherein said at least one wheel comprises a lock lever operable to selectively permit rotation of said at least one wheel, and alternatively prevent rotation of said least one wheel.

7. The limb support device of claim 1, wherein said at least one support is supported eccentrically relative to a length of said base.

8. The limb support device of claim 1, wherein said cradle is movably mounted on said base, and wherein said device comprises a locking mechanism for selectively locking said cradle at a desired angular position within a range of angular positions relative to said base.

9. The limb support device of claim 8, wherein said cradle is movably mounted to said at least one of support.

10. The limb support device of claim 1, wherein said cradle is generally concave in shape.

11. The limb support device of claim 10, wherein said cradle is U-shaped.

12. The limb support device of claim 1, wherein an inner surface of said cradle is lined with a cushioning material.

13. The limb support device of claim 12, further comprising a cover, wherein said cover and one of said cradle and said cushioning material comprise complementary matable fasteners for releasably attaching said cover to said cradle.

14. A limb support device comprising:
a rigid base;
at least one support mounted to the base to extend upwardly therefrom, said at least one support being adjustable in length and being selectively fixable at a desired length; and
a cradle mounted on said at least one support, said cradle defining a surface configured to support a limb of a patient.

15. The limb support device of claim 14, wherein said at least one support comprises one of a hydraulic cylinder and a pneumatic cylinder.

16. The limb support device of claim 14, further comprising at least one wheel supported on said base and extending in a direction opposite to that of said at least one support.

17. The limb support device of claim 14, wherein said at least one support is supported eccentrically relative to a length of said base.

18. The limb support device of claim 17, wherein said cradle is movably mounted on said at least one support, and wherein said device comprises a locking mechanism for selectively locking the cradle at a desired angular position within a range of angular positions relative to said base.

19. The limb support device of claim 18, wherein said cradle is generally concave in shape.

20. The limb support device of claim 19, wherein an inner surface of said cradle is lined with a cushioning material.

21. The limb support device of claim 20, further comprising a cover, wherein said cover and one of said cradle and said cushioning material comprise complementary matable fasteners for releasably attaching said cover to said cradle.

22. A limb support device comprising:
a rigid base;
at least one support mounted to the base to extend upwardly therefrom, said at least one support comprising at least one of a hydraulic cylinder and a pneumatic cylinder selectively adjustable in length and fixable at a desired length, said at least one support being supported eccentrically relative to a length of said base; and
a cradle mounted on said at least one support, said cradle defining a concave surface configured to support a limb of a patient.

23. The limb support device of claim 22, further comprising at least one wheel supported on said base and extending in a direction opposite to that of said at least one support.

24. The limb support device of claim 22, wherein said cradle is movably mounted on said at least one support, and wherein said device comprises a locking mechanism for selectively locking the cradle at a desired angular position within a range of angular positions relative to said base.

25. The limb support device of claim 24, wherein an inner surface of said cradle is lined with a cushioning material.

26. The limb support device of claim 25, further comprising a cover, wherein said cover and one of said cradle and said cushioning material comprise complementary matable fasteners for releasably attaching said cover to said cradle.

27. A limb support device comprising:
a rigid base;
a cradle supported on said rigid base, said cradle being generally concave in shape and defining a surface configured to support a limb of a patient, said cradle being supportable in more than one position relative to said rigid base.
